(19) United States
(12) Patent Application Publication
(10) Pub. No.: US 2014/0121620 A1
(43) Pub. Date: May 1, 2014

(54) ADHESIVE WOUND PATCH

(71) Applicant: TATUNG COMPANY, Taipei (TW)

(72) Inventor: Tsui-Shan Hung, Taipei (TW)

(73) Assignee: TATUNG COMPANY, Taipei (TW)

(21) Appl. No.: 13/761,155

(22) Filed: Feb. 7, 2013

(30) Foreign Application Priority Data
Oct. 29, 2012 (TW) 101220897

(51) Int. Cl. A61F 13/02

(52) U.S. Cl. CPC A61F 13/0206; A61F 13/0213; A61F 13/0226
USPC 604/368; 604/385.05; 604/369; 604/376

(57) ABSTRACT

An adhesive wound patch is provided. The adhesive wound patch includes a covering layer, a dressing layer, a releasing paper and a supporting layer. A pressure sensitive adhesive is coated on an inner surface of the covering layer, and the pressure sensitive adhesive with stickiness may adhere to the surrounding skin tightly. The dressing layer covers the pressure sensitive adhesive, the covering layer includes waterproof-breathable material, and the dressing layer includes hydro-absorbent material. The releasing paper includes separated first and second portions, and covers the entire covering layer. The supporting layer sticks to the covering layer, and has an ear portion, which is in the middle part of one side of the supporting layer and protrudes beyond the edge of the covering layer. After the dressing layer is adhered to the wound, the supporting layer is separated from the covering layer by tearing off the ear portion.

### CROSS-REFERENCE TO RELATED APPLICATION

This application claims the priority benefit of Taiwan application serial no. 101220897, filed on Oct. 29, 2012. The entirety of the above-mentioned patent application is hereby incorporated by reference herein and made a part of this specification.

### BACKGROUND OF THE INVENTION

1. Field of the Invention

The invention relates to a patch, and more particularly, to an adhesive wound patch.

2. Description of Related Art

Conventional wound dressings are mainly gauze and cotton, but the dressing materials are not adhesive and can not absorb large amounts of wound exudates, and therefore have the disadvantages of easily falling off of the wound and limiting the user's movement, and additionally require an adhesive tape or a protective film to fix. Since dressing materials such as gauze and cotton have the above disadvantages, products such as advanced dressings and first aid adhesives have emerged. In particular, artificial skin and hydrogel may absorb larger amounts of wound exudates, and may keep the wound moist to accelerate wound healing, and are therefore widely used to replace dressing materials such as gauze and cotton. Moreover, artificial skin and hydrogel may be combined with the covering layer to form a dressing patch with stickiness, and the dressing patch is therefore suitable for dressing wounds.

In general, first aid adhesives and dressing patches include covering layers, medical grade pressure sensitive adhesives coated on the covering layers, wound dressings such as gauze, cotton, artificial skin or hydrogel stuck to the pressure sensitive adhesives, and releasing papers used to prevent contamination to wound dressings and used as support layers. When using, the releasing paper is first removed, and the dressing patch adheres to the skin. In particular, in order to achieve the goals of even adherence and softness, the covering layers of the dressing patches are usually soft materials such as polyurethane films, polyethylene films, polypropylene films, or waterproof fabric that have the advantage of softness, but are not supportive. Therefore, when tearing off the releasing paper, the dressing patch tends to become uneven, and as a result may not properly adhere to the skin, or may require adjustment by hand, thus increasing the probability of contamination by touching the dressing.

### SUMMARY OF THE INVENTION

The invention provides an adhesive wound patch with improved convenience of use.

The invention provides an adhesive wound patch, including a covering layer, a dressing layer, a releasing paper, and a supporting layer. A pressure sensitive adhesive is coated on an inner surface of the covering layer, and the pressure sensitive adhesive with stickiness may adhere to the wound tightly. The dressing layer partially covers the pressure sensitive adhesive, wherein the dressing layer includes a hydro-absorbent material and the covering layer includes a waterproof-breathable material. The releasing paper includes a first portion and a second portion which are separated, and covers the entire covering layer by sticking to an inner surface thereof The releasing paper may be released from the covering layer. The supporting layer sticks to an outer surface of the covering layer, and has an ear portion, which is disposed in the middle part of one side of the supporting layer and protrudes beyond the edge of the covering layer. After the dressing layer is adhered to the wound, the supporting layer is separated from the covering layer by tearing off the ear portion.

In an embodiment of the invention, the ear portion of the supporting layer has an ω shape or a hill shape.

In an embodiment of the invention, the releasing paper is separated from the covering layer by tearing off a part of the first portion or the second portion not stuck to the covering layer.

In an embodiment of the invention, the dressing layer includes hydrogel, acrylic gel, PU gel, PU foam, or sodium carboxymethyl cellulose.

In an embodiment of the invention, one of the first portion and the second portion of the releasing paper has an ear portion overlapped with the ear portion of the supporting layer.

Based on the above, the invention provides an adhesive wound patch having a supporting layer supporting the covering layer, so that the covering layer may adhere evenly to the skin with ease to prevent the covering layer from wrinkling and forming gaps during use. Moreover, after the covering layer covers the wound, the supporting layer and the covering layer may be separated by tearing off the ear portion of the supporting layer. In this way, the wound is completely covered, and the probability of wound exposure is lowered, therefore the convenience of the adhesive patch and the applicability of the dressing on the wound are significantly increased.

In order to make the aforementioned and other features and advantages of the invention more comprehensible, several embodiments accompanied with figures are described in detail below.

### BRIEF DESCRIPTION OF THE DRAWINGS

The accompanying drawings are included to provide a further understanding of the invention, and are incorporated in and constitute a part of the specification. The drawings illustrate embodiments of the invention and, together with the description, serve to explain the principles of the invention.

FIG. 1A is an exploded view of an adhesive wound patch according to an embodiment of the invention.

FIG. 1B is a schematic diagram of an adhesive wound patch according to an embodiment of the invention.

FIG. 2A is an exploded view of an adhesive wound patch according to an embodiment of the invention.

FIG. 2B is a schematic diagram of an adhesive wound patch according to an embodiment of the invention.

### DESCRIPTION OF THE EMBODIMENTS

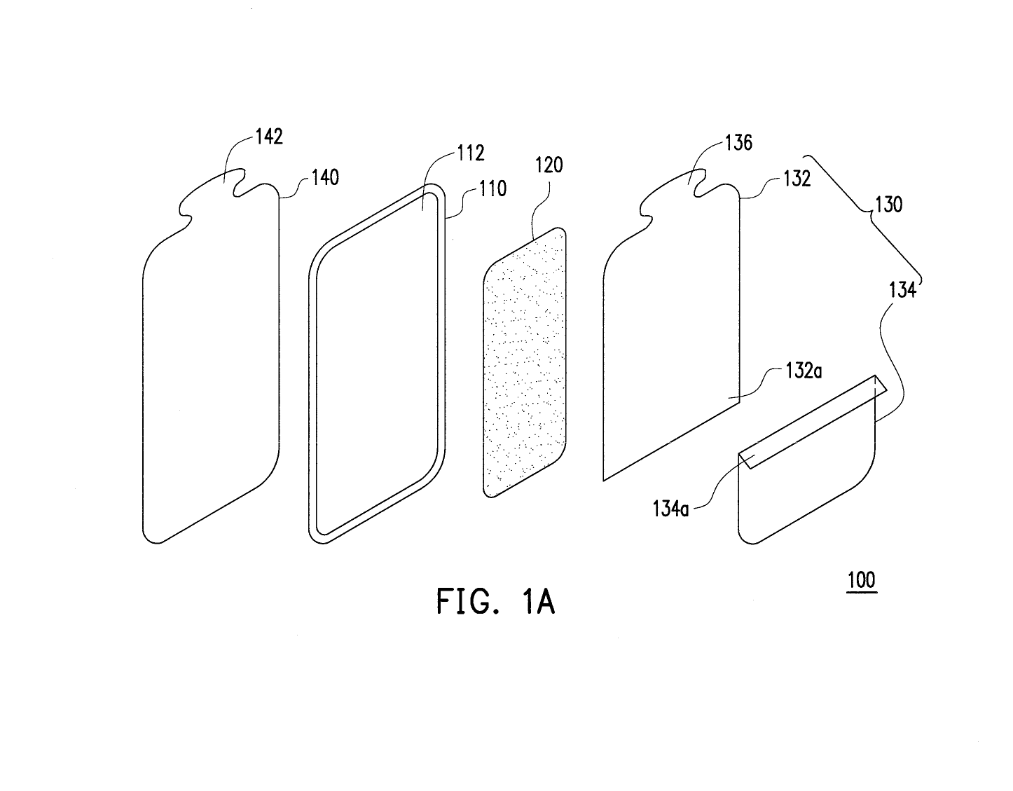

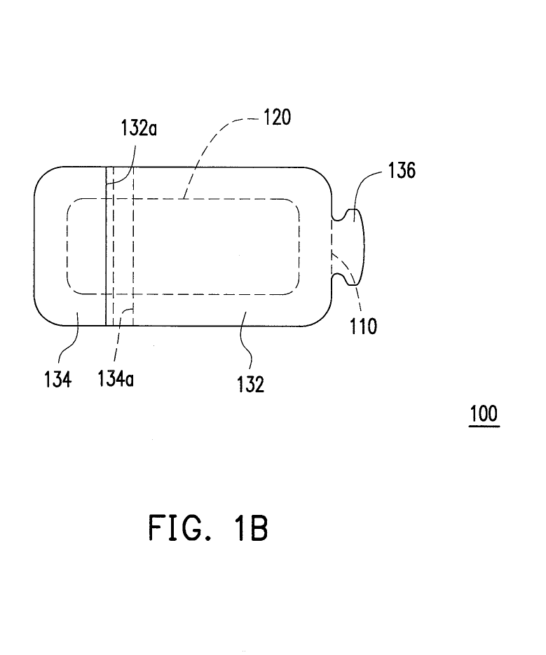

FIG. 1A is an exploded view of an adhesive wound patch according to an embodiment of the invention, and FIG. 1B is a schematic diagram of an adhesive wound patch according to an embodiment of the invention. Referring to FIG. 1A and FIG. 1B, an adhesive patch 100 includes a covering layer 110, a dressing layer 120, a releasing paper 130, and a supporting layer 140. A pressure sensitive adhesive 112 is coated on an inner surface of the covering layer 110, and the pressure sensitive adhesive 112 with stickiness may adhere to the wound tightly. The covering layer 110 includes a waterproof-breathable material. In the embodiment, the covering layer 110 is composed of waterproof-breathable material. The pressure sensitive adhesive 112 is, for instance, coated on the entire inner surface of the covering layer 110. Moreover, in the embodiment, the adhesive patch 100 is, for instance, composed of the covering layer 110, the dressing layer 120, the releasing paper 130, and the supporting layer 140. In the embodiment, the adhesive patch 100 is, for instance, roughly rectangular, but the invention is not limited thereto. That is, in other embodiments, the adhesive patch 100 may also be roughly a square or other appropriate shapes.

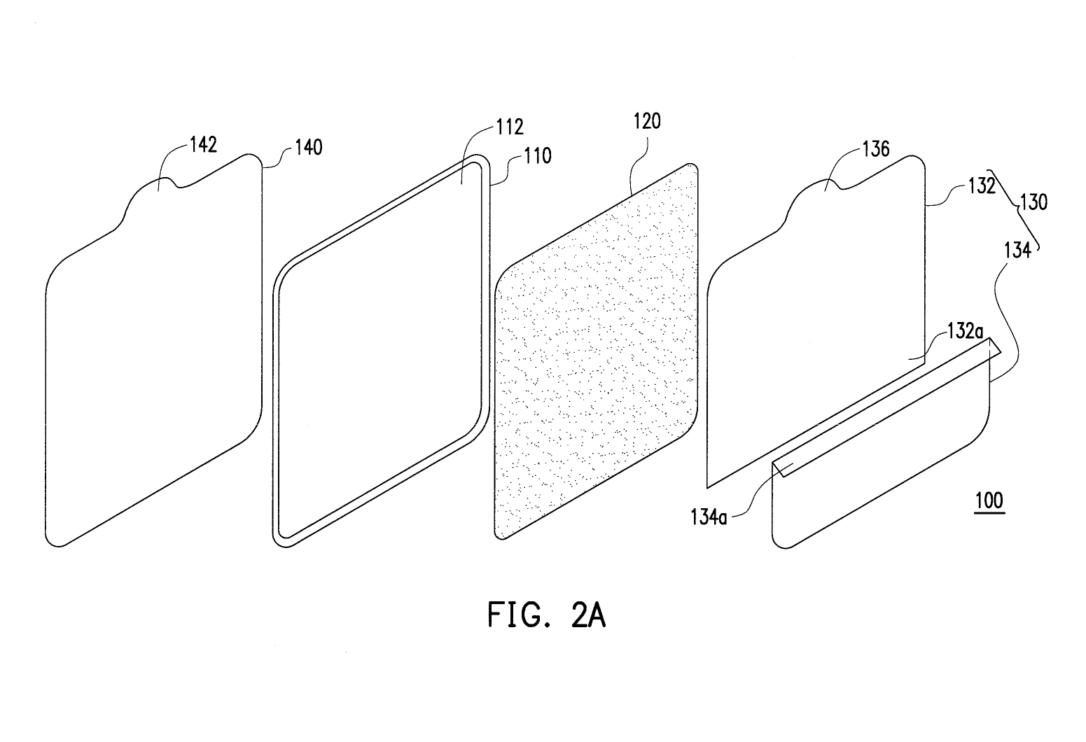

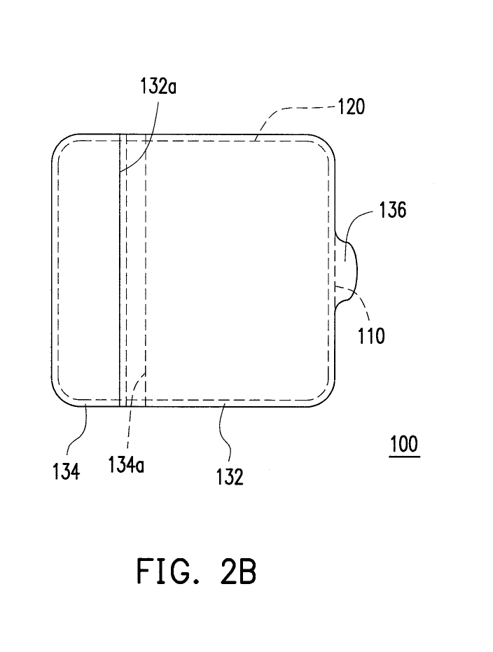

The dressing layer 120 partially covers the pressure sensitive adhesive 112, wherein the dressing layer 120 includes a hydro-absorbent material. In the embodiment, the dressing layer 120 is composed of hydro-absorbent material. The dressing layer 120 may be hydrogel, acrylic gel, PU gel, PU foam, or sodium carboxymethyl cellulose. It should be mentioned that, since the dressing layer 120 has a waterproof-breathable characteristic, when the adhesive patch 100 covers the wound, the heat generated in the wound may be released to the outside, and at the same time, the wound is protected from getting wet. Therefore, the wound may remain dry during healing to reduce the probability of an infection. Moreover, in the embodiment, the dressing layer 120 is disposed, for instance, in the middle portion of the pressure sensitive adhesive 112, and therefore the pressure sensitive adhesive 112 surrounds the dressing layer 120. In other words, when the adhesive patch 100 covers the wound, the dressing layer 120 in the middle is the part contacting the wound, and the surrounding pressure sensitive adhesive 112 is the part contacting the surrounding skin of the wound. In this way, the pressure sensitive adhesive 112 surrounding the dressing layer 120 helps to fix the adhesive patch 100 on the wound to prevent sliding. Of course, in other embodiments, as shown in FIG. 2A and FIG. 2B, the dressing layer 120 may also cover the entire surface of the pressure sensitive adhesive 112, but the invention is not limited thereto.

The releasing paper 130 includes a first portion 132 and a second portion 134 that are separated, and covers the entire covering layer 110 by sticking to the inner surface of the covering layer 110, and the releasing paper 130 may be released from the covering layer 110. In other words, the releasing paper 130 completely covers the dressing layer 120 and the pressure sensitive adhesive 112 on the inner surface of the covering layer 110 to prevent the dressing layer 120 and the pressure sensitive adhesive 112 from outside contamination or losing stickiness. The shape of the releasing paper 130 is, for instance, the same as the shape of the supporting layer 140. For the convenience of releasing, the releasing paper 130 is composed of a first portion 132 and a second portion 134, wherein the first portion 132 and the second portion 134 have edge portions 132a and 134a not stuck to the covering layer 110. The edge portion 132a of the first portion 132 is, for instance, above the edge portion 134a of the second portion 134, and the edge portion 134a of the second portion 134 is, for instance, a folded portion. In this way, the first portion 132 and the second portion 134 may be separated from the covering layer 110 by tearing off the edge portions 132a and 134a. In general, since the middle portion of the dressing layer 120 contacts the wound, contamination to the portion thereof should be avoided. Therefore, to prevent the finger from contaminating the middle portion of the dressing layer 120 when tearing off the releasing paper 130, the releasing paper 130 is designed to be, for instance, ¼ and ¾, ⅖ and ⅗, or other proportions of the first portion 132 and the second portion 134, but the invention is not limited thereto.

The supporting layer 140 is stuck to the outer surface of the covering layer 110 and has an ear portion 142 protruding beyond the edge of the covering layer 110. The ear portion 142 is disposed in the middle part of one side of the supporting layer 140, and when the dressing layer 120 is adhered to the wound, the supporting layer 140 is separated from the covering layer 110 by tearing off the ear portion 142. As shown in FIG. 1A and FIG. 1B, in the embodiment, the ear portion 142 has, for instance, an ω shape, and the ear portion 142 is disposed, for instance, in the middle part of the short side of the supporting layer 140. In another embodiment, as shown in FIG. 2A and FIG. 2B, the ear portion 142 may also have a hill shape with a smooth and flat top. In other words, the invention does not limit the shape of the ear portion 142, providing the ear portion 142 is disposed in the middle part of either side of the supporting layer 140 and has a shape convenient for stripping for the user. Moreover, in the embodiment, one of the first portion 132 and the second portion 134 of the releasing paper 130 has an ear portion 136 overlapped with the ear portion 142 of the supporting layer 140. In other words, the releasing paper 130 and the supporting layer 140 may in essence be, for instance, completely overlapped and having the same exact shapes, but the invention is not limited thereto. In other words, in other embodiments, the releasing paper 130 may also be overlapped with only the covering layer 110 and not have the ear portion 142. It should be mentioned that, from looking at the overall appearance of the adhesive patch 100, since the ear portion 142 of the supporting layer 140 protrudes out, the user may easily recognize the location thereof, benefiting the use of the adhesive patch 100.

Moreover, in an embodiment, the outer surfaces of the releasing paper 130 and the supporting layer 140 may have printed indicators of the release order and the release direction. For instance, the releasing paper 130 has label 1 and the supporting layer 140 has label 2 to indicate the release order, and the releasing paper 130 and the supporting layer 140 have printed arrows thereon to indicate the release direction. In this way, the user is able to release the releasing paper 130 and the supporting layer 140 in order, and correctly use the adhesive patch 100.

Referring further to FIG. 1A and FIG. 1B, the steps of using the adhesive patch of the invention are as follows: first, by holding the edge portions 132a and 134a of the first portion 132 and the second portion 134 of the releasing paper 130, the first portion 132 and the second portion 134 are torn off to facilitate the exposed dressing layer 120 adhere to the wound on the skin. In the step, only one of the first portion 132 and the second portion 134 is torn (such as the longer portion), or portions of both are torn by tearing the edge portions 132a and 134a at the same time to expose the dressing layer 120 in the middle of the covering layer 110.

Then, the remaining portions of first portion 132 and the second portion 134 are stripped. It should be mentioned that, the dressing layer 120 surrounded by the pressure sensitive adhesive 112 does not contact the finger when the remaining portions of first portion 132 and the second portion 134 are stripped, and the pressure sensitive adhesive 112 and the supporting layer 140 thereon adhere evenly and completely to the skin.

Then, by holding the ear portion 142 of the supporting layer 140 with the fingers, the supporting layer 140 may be stripped by gently tearing off the ear portion 142, leaving only the covering layer 110 attached to the wound and the surrounding skin.

In summary, the invention provides an adhesive wound patch including a supporting layer supporting the covering layer, so that the covering layer may adhere evenly to the skin with ease. In this way, wrinkles and gaps generated during use due to the covering layer being too soft may be avoided. As a result, the covering layer is able to completely cover the wound, and since the movement of the covering layer is prevented, a secondary injury or contamination to the wound is avoided. Moreover, when the covering layer covers the wound, the supporting layer may be removed by gently tearing off the ear portion of the supporting layer, leaving the covering layer on the wound and the surrounding skin. In this way, the wound is completely covered by the covering layer, and the probability of wound exposure is reduced. Therefore, the convenience of the adhesive patch and the applicability of the dressing on the wound are significantly increased.

Although the invention has been described with reference to the embodiments thereof, it will be apparent to one of the ordinary skills in the art that modifications to the described embodiments may be made without departing from the spirit of the invention. Accordingly, the scope of the invention will be defined by the attached claims not by the above detailed description.

What is claimed is:

1. An adhesive wound patch, comprising:
a covering layer, wherein a pressure sensitive adhesive is coated on an inner surface of the covering layer, the pressure sensitive adhesive with stickiness is able to adhere to a wound tightly, and the covering layer comprises a waterproof-breathable material;
a dressing layer partially covering the pressure sensitive adhesive, wherein the dressing layer comprises a hydro-absorbent material;
a releasing paper, comprising a first portion and a second portion which are separated, wherein the releasing paper covers the entire covering layer by sticking to an inner surface thereof, and is releasable from the covering layer; and
a supporting layer sticking to an outer surface of the covering layer and having an ear portion protruding beyond the edge of the covering layer, wherein the ear portion is disposed in a middle part of one side of the supporting layer, and after the dressing layer is adhered to the wound, the supporting layer is separated from the covering layer by tearing off the ear portion.

2. The adhesive wound patch of claim 1, wherein the ear portion of the supporting layer has an ω shape or a hill shape.

3. The adhesive wound patch of claim 1, wherein the releasing paper is separated from the covering layer by tearing off a part of the first portion and the second portion not stuck to the covering layer.

4. The adhesive wound patch of claim 1, wherein the dressing layer comprises hydrogel, acrylic gel, PU gel, PU foam, or sodium carboxymethyl cellulose.

5. The adhesive wound patch of claim 1, wherein one of the first portion and the second portion of the releasing paper has an ear portion overlapped with the ear portion of the supporting layer.
